(19) United States
(12) Patent Application Publication
(10) Pub. No.: US 2013/0260384 A1
(43) Pub. Date: Oct. 3, 2013

(54) METHOD FOR DETERMINING CANCER PROGNOSIS AND PREDICTION WITH CANCER STEM CELL ASSOCIATED GENES

(71) Applicant: UNIVERSITY OF SOUTHERN CALIFORNIA, Los Angeles, CA (US)

(72) Inventor: Amir GOLDKORN, Beverly Hills, CA (US)

(73) Assignee: UNIVERSITY OF SOUTHERN CALIFORNIA, Los Angeles, CA (US)

(21) Appl. No.: 13/853,828

(22) Filed: Mar. 29, 2013

Related U.S. Application Data
(60) Provisional application No. 61/618,536, filed on Mar. 30, 2012.

(51) Int. Cl. C12Q 1/68

(52) U.S. Cl. CPC C12Q 1/6886
USPC 435/6.12; 435/7.1; 702/19

(57) ABSTRACT

Disclosed herein is a general method of formulating a cancer diagnosis or prognosis test based on a cancer stem cell (CSC)-associated gene expression signature, the method include steps of selecting a set of candidate CSC genes, identifying a subset of gene(s) as signature gene(s); and formulating a test based on measurement of the expression level of the signature genes. Also disclosed herein are exemplary diagnostic/prognostic tests formulated using the general method. In particular, provided herein is a set of signature genes for formulating a diagnostic/prognostic test of prostate cancer, including Axin2, CD44, Oct4, TACSTD2, NANOG, and CTNNB1. The present invention further provides kits, devices and systems for performing the diagnostic/prognostic test, which generally include analytical elements capable of detecting an analyte in a sample corresponding to the expression level of one or more signature genes, preferably selected from Axin2, CD44, Oct4, TACSTD2, NANOG, and CTNNB1.

### CROSS-REFERENCE TO RELATED APPLICATIONS

This application claims an invention which was disclosed in Provisional Application No. 60/618,536 filed Mar. 30, 2012, the entire content of which is hereby incorporated herein by reference.

### FIELD OF THE INVENTION

The present invention relates generally to the field of cancer prognosis by utilizing cancer stem cell associated genes. More particularly, the present invention provides a method for determining cancer prognosis or prediction with cancer stem cell associated genes.

### BACKGROUND OF THE INVENTION

Cancer has a devastating personal and societal impact. Despite medical advances, malignancy currently accounts for over 500,000 annual deaths in the U.S. alone and is associated with untold pain and suffering (1). In recent decades, vast resources have been allocated to the development of novel cancer treatments, thus shifting the therapeutic “bottle-neck” towards a new and critical need for effective biomarker strategies to better inform the selection of optimal treatments and guide their use in patients.

Among the various forms of cancers, prostate cancer (PC) is the most prevalent and the second most lethal cancer in American men (1), with the vast majority of deaths occurring due to advanced metastatic disease that has recurred after failed local therapy. In 2012, an estimated 241,740 Americans will be diagnosed with prostate cancer and 28,170 Americans will die of prostate cancer (1). Radical prostatectomy±bilateral pelvic lymph node dissection is a standard treatment for clinically localized prostate cancer (CLPC) (19).

While there are standard treatments available for prostate cancer, population-level management of the disease suffers from significant over-diagnosis in the U.S. due to wide use of PSA screening, which often leads to the diagnosis of prostate cancer of unclear clinical significance that is nonetheless treated aggressively with radical prostatectomy with all its attendant toxicities. Thus, the need for better prognostic indicators to guide appropriate management is perhaps nowhere more acutely felt than in this particular disease setting, where on the one hand patients with clinically mild disease are frequently over-treated and needlessly subjected to the side effects of prostatectomy, and on the other hand many men who undergo prostatectomy ultimately experience recurrence, progression and death from disease which could perhaps be mitigated if this aggressive course were to be known in advance.

Another problem area with the management of prostate cancer is post-treatment prognosis of recurrence. Currently used predictors of disease recurrence after radical prostatectomy include preoperative prostate-specific antigen (PSA), primary and secondary Gleason grade, extracapsular extension, positive surgical margins, seminal vesicle invasion, lymph node involvement, and treatment year (20). However, even though these variables are important predictors of outcome, there remains significant variability in the prognosis of patients with similar clinical and pathologic characteristics (20-21).

In view of the above, there is an urgent, unmet need for a new diagnostic paradigm to provide actionable information so as to enable individualized therapy planning and treatment decision making.

### SUMMARY OF THE INVENTION

Recently, molecular lesions (i.e. damages to a biological molecule resulting in reduction, loss, or gain of function) associated with clinically significant prostate cancer have been identified. For example, the loss of one or both copies of the tumor suppressor PTEN (22-23), TMPRSS2-ERG chromosome fusion (24-26), p53 mutations (22, 27), overexpression of MYC (28), alpha-methylacyl-CoA-racemase expression (29) and gene panel expression profiles (22, 30-34) have been shown to be associated with outcome. It has been theorized that these variables may potentially be used as inputs for prognosis of treatment outcome. However, no reliable method for the determination of disease recurrence exists (20-21, 35).

Current paradigm of drug development is based on animal model testing where therapies able to promote tumor shrinkage are viewed as effective. However, animals rarely provide a complete model of human disease. In particular, in mice models, the short lifespan of mice (around 2 years) makes it exceptionally difficult to study tumor relapse. Because cancer stem cells (CSCs) typically form a very small portion of a tumor, current paradigm of therapy selection may not really select for drugs that target CSCs, leaving them behind to give rise to new tumors (i.e. relapse). It stands to reason that CSC may hold the key to a new paradigm of cancer diagnosis, particularly the likelihood of cancer recurrences.

Accordingly, the present invention is based, in part, on the insight that CSC-associated genes may offer clues as to the likely outcome of a cancer patient in response to a particular therapy. The present invention is also based, in part, on the unexpected discovery of a CSC-associated gene expression signature that predicts prostate cancer recurrence.

While not intending to be bound by any particular theory, it has been proposed that tumor formation and dissemination may be caused by CSCs. Experimental evidence for the presence of CSCs has accrued steadily: Prospectively-isolated sub-populations (about 1%) of tumors were first found in acute myeloid leukemia, breast cancer and glioblastoma, and more recently reported in prostate, pancreatic, colon, and bladder cancers (2-7). CSC subpopulations continue to be identified in virtually every malignancy type. The recent discovery of highly tumorigenic, drug resistant, and invasive CSC sub-populations carries significant implications that inform and alter the way in which cancer is conceptualized and managed. In particular, this current invention is based on the novel insight that measuring the CSC phenotype in a given tumor can provide important prognostic/predictive information about disease recurrence that will improve medical decision making. Some markers used to identify CSC subpopulations appear to play a direct functional role in their invasive/metastatic phenotype. For example, CD44, a cell surface marker frequently used to identify CSC subpopulations in prostate tumors, has been shown to mediate the adhesion of prostate cancer cells to bone marrow derived endothelial cells (8), and CD44+ cancer cells with a CSC phenotype have been shown to possess many features of epithelial to mesenchymal transition (EMT), a well-studied phenomenon in which tumor cells mobilize and metastasize to distant sites (9).

In light of the above insight, one aspect of the present invention is directed to novel biomarker strategies for cancer diagnosis and prognosis utilizing CSC and CSC-associated genes. Unlike prior art methods of prostate cancer diagnostic/prognostic methods that either uses PSA or expression of genes associated with cancer cells, the present invention focuses on cancer stem cells and their associated genes.

As used herein, the term “CSC-associated gene expression signature” refers to a set of CSC-associated genes whose expression levels have been shown to have differential expression patterns between a control and comparison group of subjects. That is, these genes either independently or collectively can be shown by a statistical means that their expression pattern in the control group of subjects is predictably different from their expression pattern in the comparison group. Member genes that form a CSC-associated gene expression signature are hereinafter referred to individually as a “CSC-associated signature gene” or simply as a “signature gene”.

Embodiments in accordance with this aspect of the invention include methods for determining a CSC-associated gene expression signature useful as a biomarker for formulating a cancer diagnostic/prognostic test; methods for formulating cancer diagnostic/prognostic assay using said CSC-associated gene expression signature; and methods for determining a cancer diagnosis/prognosis for a subject using a diagnostic/prognostic assay based on a CSC-associated gene expression signature.

The process of identifying CSC-associated “signature genes”, formulating a diagnostic/prognostic test based on the signature genes, and then utilizing the diagnostic/prognostic test to assess and stratify the aggressiveness of an individual's cancer form a new paradigm for individualized prostate cancer diagnostics/prognostics and clinical management. Insofar as CSCs are increasingly being linked to relapse of other types of cancers, it will be understood by those skilled in the art that this new paradigm of CSC-associated signature gene diagnostic/prognostic may also be advantageously be applied to any number of cancers.

### 1. Methods for Determining a CSC-Associated Gene Expression Signature

Methods for determining a CSC-associated gene expression signature in accordance with embodiments of the present invention will generally include the steps of selecting a set of candidate CSC-associated genes; measuring the expression levels of these candidate genes in tissue samples obtained from a population of subjects consisting of a control group and a comparison group; analyzing the expression levels to identify one or more signature gene(s) among that candidate genes that show differential expression pattern in the control and comparison groups; and designating the identified genes as the CSC-associated gene expression signature.

Selection of the candidate CSC-associated genes may be done by literature review, screening experiments, or any other methods of gene association known in the art. In one preferred embodiment, the candidate CSC-associated gene(s) are preferably selected from the group consisting of ALDH1A1, Axin2, Bmi1, CD133, CD44a, CTNNB1/β-catenin, ITGA2/integrin α2, NANOG, Nkx3-1, Notch1, OCT 4, TACSTD2/Trop2. In another preferred embodiment, the at least one CSC-associated gene is selected from the group consisting of Axin2, NANOG, and CTNNB1. In a more preferred embodiment, the at least one CSC associated gene is Axin2. In a still more preferred embodiment, the method includes measuring the expression level of 2 or more CSC-associated genes, and more preferably the CSC-associated genes comprise Axin2, Nanog, and CTNNB1.

Gene expression levels may be measured either directly or indirectly by any currently known or future developed measurement methods. Exemplary direct measurement methods will typically be measuring the level of mRNA in the tissues. For this purpose, the gene expression measurement method may include but not limited to PCR based method such as conventional endpoint PCR, RT PCR, qPCR, RT qPCR, fluorescent based methods such as fluorescent in situ hybridization, and the like. Preferably, real-time quantitative PCR is used. The level of gene expression may also be measured indirectly by measuring other surrogate markers associated with these genes. Exemplary surrogate markers may include but not limited to proteins encoded by the genes, or any other quantifiable physical/biochemical entities that relates to the expression level of the signature genes.

The tissue sample used in accordance with the present invention preferably comprises tumor tissue. More preferably, the tissue sample is a fixed formalin tissue sample.

A preferred method of measuring the expression level of the CSC-associated gene is microdissecting areas of tissue sample comprising tumor tissue from formalin fixed paraffin embedded (FFPE) specimens, extracting RNA from the microdissected tumor tissue, and measuring an mRNA expression level of the at least one CSC-associated gene by qRT-PCR with normalization to housekeeping genes. More preferably, the methods of the present invention include measuring an expression level of the at least one CSC-associated gene by qRT-PCR measurement on freshly-resected or fresh-frozen tissue or on circulating tumor cells isolated from blood samples.

Various statistical methods may be utilized to identify genes that show differential expression pattern. In a preferred embodiment, a univariable analysis, a multivariable analysis, or both may be used. In a more preferred embodiment, Wilcoxon's test is used. In another preferred embodiment, a classification and regression tree based on recursive partitioning is used.

Cancer as used in this context refers generally to a number of different types of cancers, including, but not limited to breast, lung, colon, bladder, liver, pancreatic and brain cancer. Depending on the desired diagnostic/prognostic output, the control and comparison group may be subjects or individuals at various stages of cancers. For example, in one embodiment, the control group may be healthy subject whereas the comparison group may be subjects suffering from a particular cancer such as breast or prostate cancer. Those skilled in the art will readily understand that the choice of subjects may or may not be random, depending on the desired diagnostic/prognostic information to be obtained. FIG. 1 shows an exemplary selection process for choosing suitable subjects to be included in the final population for analysis.

In a preferred embodiment, the desired diagnostic/prognostic information is the likelihood of prostate cancer recurrence after prostatectomy. In this embodiment, the control group is preferably subjects who had prostatectomy but showed no sign of recurrence, whereas the comparison group is preferably subjects who had prostatectomy and also exhibiting signs of recurrence. More preferably, for the purpose of diagnosing/prognosing the recurrence risk of prostate cancer post prostatectomy, the signature gene(s) are selected from the group consisting of Axin2, CD44, Oct4, TACSTD2, NANOG, and CTNNB1. More preferably, the signature gene(s) are Axin2, CD44, Oct4, and TACSTD2. Another preferred set of signature gene(s) are Axin2, NANOG, and CTNNB1. Other preferred set are possible, and preferably include at least Axin2.

### 2. Methods for Formulating a Cancer Diagnostic/Prognostic Assay Based on a CSC-Associated Gene Signature

Methods for formulating a cancer diagnostic/prognostic assay in accordance with embodiments of the present invention will generally include the steps of forming a candidate classifier that takes the expression levels of a CSC-associated gene signature of a test subject as input, wherein said classifier has one or more cutpoint parameters that determines a classification for the test subject; generating a receiver operating characteristic (ROC) curve by applying the classifier to a training data set with varying values of cutpoint parameters, wherein said training data set consists of known positive and negative samples; and choosing the values of cutpoint parameters corresponding to the optimal point along the ROC curve as the final values for the classifier's cutpoint parameters.

CSC-associated gene signatures usable to formulate the assay are as described above. Assays formulated in accordance with methods described above will generally comprise a classifier optimized according to the formulation method described above.

As used herein, the term “classifier” refers to a decision making rule or algorithm that takes a set of input about a sample or test subject and then generates a classification for the sample or test subject based on the input.

It will be appreciated by those skilled in the art that any number of diagnosis/prognosis may be made so long as there is sufficient training data to train and optimize the classifier. Exemplary diagnosis/prognosis that may be made may include but not limited to likelihood of cancer, likelihood of cancer recurrence, likelihood of response to a therapy, and etc.

For example, in one embodiment, the training data set may include expression level of the signature CSC-associated genes in prostate cancer disease states serologic progression (PSA rise), metastatic hormone sensitive disease, and metastatic castration resistant disease. The cancer prognosis or cancer prediction may be for predicting a response to therapy, including at least one of androgen deprivation or androgen receptor antagonist therapy (in serologic progression or metastatic hormone sensitive disease states), and predicting response to chemotherapy or targeted agents in the castration resistant metastatic disease state.

In another preferred embodiment, the training set may include data for training the classifier to predicting prostate cancer response to therapeutic interventions ranging from surgical (e.g. prostatectomy), to radiological (e.g. radiotherapy), to hormonal (e.g. androgen deprivation therapy or androgen receptor antagonists), to chemotherapy.

Another exemplary training data set may include data for training the classifier to predicting response to therapy, disease recurrence, progression-free survival, and overall survival in the localized, locally advanced, metastatic, and chemotherapy-resistant states of other malignancy types, in particular those that already have been shown to possess subpopulations of CSC.

Still other exemplary training data set may include data for training the classifier to predict a risk level of prostate cancer recurrence of a subject after prostatectomy. Risk level is based on the expression levels of genes associated with a cancer stem-like phenotype.

Still other exemplary training data set may include data for training the classifier to predict additional clinical outcomes in prostate cancer, including overall survival and progression-free survival in the localized disease state, the serologically recurrent disease (i.e. rising serum levels of Prostate Specific Antigen, PSA), the metastatic hormone sensitive state, and the metastatic castration resistant state.

### 3. Methods for Determining a Cancer Diagnosis/Prognosis for a Subject Using a Diagnostic/Prognostic Assay Based on a CSC-Associated Gene Expression Signature

Methods for determining a cancer diagnosis/prognosis in accordance with embodiments of the present invention will generally involve using a diagnostic/prognostic assay based on a CSC-associated gene expression signature as described above. More particularly, such methods will generally include the steps of: measuring expression levels of signature genes of a CSC-associated gene expression in a test subject's tissue sample; applying a diagnostic/prognostic assay based on the CSC-associated gene signature; and determining a diagnostic/prognostic for the test subject.

CSC-associated gene expression signatures usable here are as described above. Diagnostic/prognostic assays are ones formulated as described above.

Another aspect of the present invention is directed to tools, devices, systems, and reagent kits for performing the diagnosis and prognosis strategies described above.

In one exemplary embodiment, there is provided a kit for performing a diagnostic/prognostic assay based on a CSC-associated gene signature. Kits in accordance with this embodiment of the invention will generally include one or more reagents for determining expression levels of member signature genes belonging to the CSC-associated gene signature; and an instruction insert with printed instructions for directing a user to perform the test and interpret the result.

Exemplary reagents may include but not limited to nucleic acids, antibodies, proteins, or any other molecular recognition reagents. Preferably, the kit is one for detecting mRNA levels for the signature genes. More preferably, the kit may further include other elements for facilitating the analysis, such as tissue sample preparation reagents including mRNA purification reagents, oligo-primers for hybridizing to the mRNA of signature genes, cDNA synthesizing enzymes, and other stabilizing reagents. The kit may further include reaction containers such as micro-titer plates, or gene chips. In other preferred embodiments

The kits, devices and systems will generally include a sample analysis element for determining the expression level of at least one signature gene. Any analytical devices, instrumentation, and systems known in the art may be adapted or configured to meet the purpose of the present invention so long as the device or system are configured to be able to detect the expression level of at least one signature gene as described above. In a preferred embodiment, the kits, devices, and systems are configured to be able to detect the expression level of at least one gene selected from the group consisting of Axin2, CD44, Oct4, TACSTD2, NANOG, and CTNNB1. More preferably, kits, devices and systems are configured to be able to simultaneously detect the combination of genes Axin2, CD44, Oct4, TACSTD2; or Axin2, NANOG, and CTNNB1; or both combinations.

Embodiments in accordance to the various aspects of the invention will have at least the following advantages. (1) CSC-associated expression patterns disclosed herein describe a gene expression signature that predicts cancer recurrence in men who undergo prostatectomy for localized disease; (2) the gene signature is based on a large cohort of specimens from a well-annotated archive; and (3) it employs fully quantitative (RT-PCR) measures of CSC associated genes or alternative detection methods for these genes (e.g. antibody-mediated detection of the proteins encoded by these genes). The prognostic and predictive methods of the present disclosure are valuable tools in determining whether patient will experience a positive or negative treatment outcome independent of treatment modality.

Other aspects and advantages of the present invention will become apparent from the following detailed description, the accompanying drawings, and the appended claims.

### BRIEF DESCRIPTION OF THE DRAWINGS

FIG. 1 Sample/patient selection process for our nested case-control study of CSC-associated gene expression measurement in men who underwent radical prostatectomy for localized prostate cancer at USC/Norris.

FIG. 2 Recursive partitioning (“CART”) diagram representing the recursive partitioning analysis performed to identify any multiple-gene associations that are associated with disease recurrence.

### DETAILED DESCRIPTION

### Definition

Unless otherwise indicated herein, all terms used herein have the meanings that the terms would have to those skilled in the art of the present invention. Practitioners are particularly directed to current textbooks for definitions and terms of the art. It is to be understood, however, that this invention is not limited to the particular methodology, protocols, and reagents described, as these may vary

The term “cancer stem cells” as used herein generally refers to pluripotent cells with the capacity to differentiate and give rise to entire new tumors, much the same as normal tissue stem cells are able to differentiate and regenerate normal tissues. CSC are generally characterized by a long relative life span, activation of pathways necessary for self-renewal (e.g. Wnt/β-catenin, Notch, Shh, BMI1), chemotherapy resistance, and high tumorigenicity relative to unselected tumor cells.

As used herein, the term “cancer stem cell associated genes” refers to genes that are frequently found to be differentially expressed (at higher or lower levels in cancer stem cells) relative to the overall expression level in the tumor. In general, these are genes frequent involved in a self-renewing progenitor phenotype.

The term “prognosis” as used herein may refer to the prediction of the likelihood of cancer-attributable death or progression, including recurrence, metastatic spread, and drug resistance, of a cancer, typically prostate cancer.

The term “prediction” as used herein may refer to the likelihood that a patient will have a particular clinical outcome, whether positive or negative, following treatment with surgery or chemotherapy or a combination of both. The predictive methods of the present disclosure can be used clinically to make treatment decisions by choosing the most appropriate treatment modalities for any particular patient. The predictive methods of the present disclosure are valuable tools in predicting if a patient is likely to respond favorably to a treatment regimen, such as chemotherapy, surgical intervention, or both.

The term “clinical outcome” as determined by a prognostic marker refers to disease course independent of specific therapeutic intervention and is usually measured in terms of disease progression and/or overall survival and/or disease related mortality. Any biomarker that provides information independent of specific therapeutic intervention is a prognostic marker.

The term “clinical response” can be assessed using any endpoint indicating a response to treatment in a patient, including, without limitation, (1) inhibition or progression, to some extent, of tumor growth, including slowing down and complete growth arrest or acceleration; (2) reduction or increase in the number of tumor cells; (3) reduction in tumor size; (4) inhibition (i.e., reduction, slowing down or complete stopping) of lack thereof of tumor cell infiltration into adjacent peripheral organs and/or tissues; (5) inhibition or lack thereof of metastasis; (6) enhancement of lack thereof of anti-tumor immune response, possibly resulting in regression or rejection of the tumor; (7) relief or lack thereof, to some extent, of one or more symptoms associated with the tumor; (8) increase or decrease in the length of survival following treatment; (9) decreased or increased mortality at a given point of time following treatment; and/or (10) recurrence or lack thereof of disease following a treatment regimen. Clinical response may also be expressed in terms of various measures of clinical outcome. Positive or negative clinical response can be considered in the context of an individual's outcome relative to an outcome of a population of patients having a comparable clinical diagnosis, and can be assessed using various endpoints such as an increase in the duration of Recurrence-Free interval (RFI), an increase in the time of survival as compared to Overall Survival (OS) in a population, an increase in the time of Disease-Free Survival (DFS), an increase in the duration of Distant Recurrence-Free Interval (DRFI), and the like. An increase in the likelihood of positive clinical response corresponds to a decrease in the likelihood of cancer recurrence. Any marker that indicates response to particular therapy is a predictive marker.

This invention provides a method for predicting prostate cancer prognosis based on gene expression, and in particular based on the expression of genes related to a progenitor, cancer stem-like state.

In one aspect, the current invention provides a method for determining the risk of prostate cancer recurrence (PSA rise or clinical metastases) after radical prostatectomy for localized prostate cancer.

A method of cancer prognosis or prediction in a test subject comprises: Providing a tissue sample from the test subject, the tissue sample comprising a population of cancer cells; measuring an expression level of at least one cancer stem cell associated (CSC-associated) gene in the tissue sample; comparing the expression level of the at least one CSC-associated gene measured in the test sample relative to a reference expression level for the same at least one CSC associated gene, the reference expression level being derived from a cohort of patients having a comparable diagnosis; and based on the comparison, identifying the test subject as either likely or unlikely to experience a clinical response.

The cancer is selected from the group consisting of prostate, breast, lung, colon, bladder, liver, pancreatic and brain cancer and the tissue sample preferably comprises at least a portion of tumor tissue thereof.

In another embodiment, a method for predicting the likelihood of recurrence of prostate cancer following postatectomy in a test subject, the method comprising: providing a tissue sample from the test subject, the tissue sample comprising a population of prostate cancer cells; measuring an expression level of at least one cancer stem cell associated (CSC-associated) gene in the tissue sample; comparing the expression level by comparing the expression level of the at least one CSC-associated gene measured in the test sample to a reference expression level for the at least one CSC associated gene, the reference expression level being derived from a cohort of patients who have not experienced recurrence of prostate cancer following prostatectomy; and identifying the test subject as either likely to experience a recurrence of prostate cancer based on the comparison.

The tissue sample used in accordance with the present invention preferably comprises tumor tissue. More preferably, the tissue sample is a fixed formalin tissue sample.

The at least one CSC-associated gene is preferably selected from the group consisting of ALDH1A1, Axin2, Bmi1, CD133, CD44a, CTNNB1/β-catenin, ITGA2/integrin α2, NANOG, Nkx3-1, Notch1, OCT 4, TACSTD2/Trop2. In a preferred embodiment, the at least one CSC-associated gene is selected from the group consisting of Axin2, NANOG, and CTNNB1. In another preferred embodiment, the at least one CSC associated gene is Axin2. In another preferred embodiment, the method includes measuring the expression level of 2 or more CSC-associated genes, and more preferably the CSC-associated genes comprise Axin2, Nanog, and CTNNB1.

A preferred method of measuring the expression level of the CSC-associated gene is microdissecting areas of tissue sample comprising tumor tissue from formalin fixed paraffin embedded (FFPE) specimens, extracting RNA from the microdissected tumor tissue, and measuring an mRNA expression level of the at least one CSC-associated gene by qRT-PCR with normalization to housekeeping genes. More preferably, the methods of the present invention include measuring an expression level of the at least one CSC-associated gene by qRT-PCR measurement on freshly-resected or fresh-frozen tissue or on circulating tumor cells (CTCs) isolated from a patient blood sample.

Another embodiment of the present invention comprises measuring the expression level of the at least one CSC-associated genes in prostate cancer disease states including serologic progression (PSA rise), metastatic hormone sensitive disease, and metastatic castration resistant disease. The cancer prognosis or cancer prediction may be for predicting a response to therapy, including at least one of androgen deprivation or androgen receptor antagonist therapy (in serologic progression or metastatic hormone sensitive disease states), and predicting response to chemotherapy or targeted agents in the castration resistant metastatic disease state.

One example of CSC-associated gene signature analysis is directed to localized prostatectomy specimens and will be described below.

### Demonstrative Experiments

The following example is provided in order to demonstrate and further illustrate certain embodiments and aspects of the present invention. They are not to be construed as limiting the scope thereof. Specifically, it will be understood by those skilled in the art that the examples may be modified based on the general procedures and methods described herein to other clinical response, cancer and disease state associated with each cancer.

In connection with this study, a large cohort was used to identify a subset of CSC-associated genes (in this specific example, Axin2, Nanog, CNNTB1) whose mRNA expression level is significantly associated with recurrence status after prostatectomy for localized prostate cancer. Specifically: A subset of independent single genes (Axin2, CD44, Oct4, TACSTD2) whose median normalized mRNA expression levels were significantly different between recurrent and non-recurrent patients (univariable analysis) A subset of inter-dependent genes (Axin2, Nanog, CTNNB1) that generated cutpoints (based on CART recursive partitioning) which significantly discriminated between recurrent and non-recurrent patients (multivariable analysis).

This procedure may be used in connection with this embodiment and others as follows. First, Generate an ROC curve based on the prognostic sensitivity and specificity of selected CSC-associated gene(s) (i.e. the 3 genes identified in this example i.e. their ability to discriminate recurrence vs. non-recurrence in this cohort). Second, select the optimal point along the ROC curve corresponding to expression value of selected CSC-associated gene(s) (e.g. Axin2/Nanog/CTNNB1 with greatest sensitivity and specificity. Third, extract RNA or protein from each tumor specimen in this cohort, and use qRT-PCR or other known procedure to measure the expression level of selected CSC-associated gene (e.g. Axin2, Nanog, and CTNNB1). Fourth, use the expression value identified in second step (e.g. Axin2/Nanog/CTNNB1 expression value) to determine whether each patient/specimen in this new cohort will have the clinical response (e.g. disease recurrence) or not.

Study Design, Setting, and Participants.

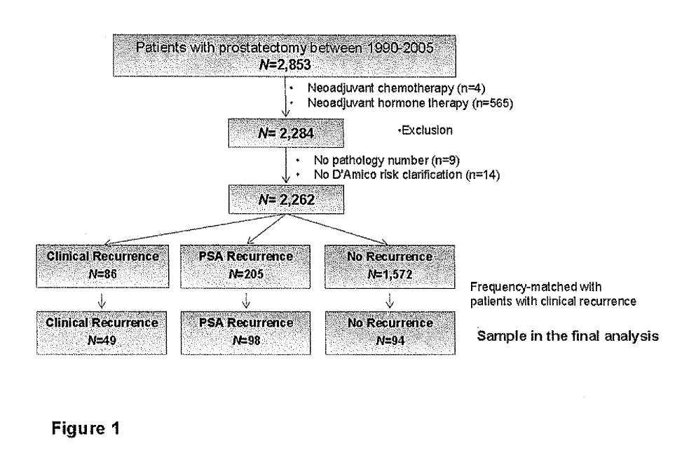

FIG. 1 shows the selection process for this nested case-control study. We identified 1468 men who underwent open radical prostatectomy and bilateral pelvic lymph node dissection for CLPC at the University of Southern California in a prospectively maintained Institutional Review Board-approved database (36). Cases were men who underwent surgery and experienced disease recurrence (biochemical or clinical). Controls were men who underwent surgery and did not experience disease recurrence (biochemical or clinical). Biochemical recurrence was defined as a rise in PSA above the contemporary undetectable level (<0.3 ng/ml from July 1988 to July 1994; <0.05 ng/ml from July 1994 to March 2005; and <0.03 ng/ml from March 2005 to present) and was verified by 2 consecutive rises in men with a postoperative undetectable PSA, with an interval of 3-4 months between PSA assessments. Clinical recurrence was defined as either palpable local disease on biopsy or distant recurrence confirmed by imaging studies including magnetic resonance imaging, computed tomography, bone scan, or chest radiography. We aimed to select 2 cases for every control. Controls were matched to cases by D'Amico risk group. The final study cohort included 276 men patients treated over nearly 20 years.

Selection of Cancer Stem-Like Cell Genes.

The selection of CSC associated genes was based on the results of a comprehensive literature search. PubMed was searched for original research articles reporting on CSC-associated progenitor gene transcripts in cancer populations. We also reviewed the reference lists of CSC review articles. Table 1 lists 12 candidate CSC-associated genes (ALDH1A1, Axin2, Bmi1, CD133, CD44a, CTNNB1/β-catenin, ITGA2/integrin α2, NANOG, Nkx3-1, Notch1, OCT 4, TACSTD2/Trop2) selected for gene expression analysis.

Sample Preparation.

All radical prostatectomy specimens collected at the time of surgery were processed expeditiously within the Department of Pathology and stored in an institutional biospecimen bank. Formalin-fixed paraffin-embedded (FFPE) tumor blocks were reviewed for quality and tumor content by dedicated genitourinary Pathologists (LW, SEM). Ten micrometer thick sections were obtained from identified areas with the highest tumor concentration and re-reviewed to ensure matching of Gleason Score (GS) to the GS reported in the prior archived pathology reports. Tumor tissue specimens were deidentified and sent in a coded manner to Response Genetics Inc. (RGI, Los Angeles, Calif.) for mRNA gene expression analysis.

mRNA Gene Expression Levels.

For this embodiment, mRNA cancer stem-like cell gene expression analysis was performed at RGI by blinded investigators who were unable to distinguish between cases and controls. Briefly, areas of tumor were microdissected from the slides, total RNA was extracted, and the genes of interest were amplified and measured by qRT-PCR and normalized to internal housekeeping genes as previously described (37).

Although in this embodiment the mRNA extraction and qRT-PCR were performed at RGI, the methods described in this invention do not rely upon nor are specific to those used at RGI. Currently, there are multiple commercially available kits (Qiagen, Life-Tech, others) for mRNA extraction from FFPE tumor tissue, and these kits can be used as effectively to extract, measure and compare CSC-associated gene expression by qRT-PCR from a given set of tumor specimens.

Statistical Analysis.

Recurrence status was the primary endpoint in the current embodiment. Men with biochemical recurrence and clinical recurrence were labeled as recurrence whereas men who remained recurrence-free were labeled as non-recurrence. Quantitative reverse transcriptase-PCR analyses yield values that were expressed as ratios between two absolute measurements: the gene of interest and internal reference gene, b-actin. All gene expression levels were continuous but the distribution was not compatible to the normality. The mRNA levels of CSC associated genes between recurrence and non-recurrence groups were compared using the univariable Wilcoxon test. This data was summarized and reported as the median (range) for each group. In addition, a classification and regression tree (CART) method based on recursive partitioning (RP) was used to explore gene expression variables for identifying homogenous subgroups for recurrence. The RP analysis is a nonparametric statistical method for modeling a response variable and multiple predictors. The analysis includes two essential processes: tree-growing and tree pruning. The RP analysis included all patients with any gene mRNA levels available (n=249). To adjust for multiple comparisons and control the false positive rate, bootstrap internal validation was performed for both the univariable and multivariable analysis. One hundred bootstrap samples of 249 observations each were drawn from the original cohort using simple random sampling with replacement. The number of simulations with a significant p value (P<0.05) in the univariate analysis or selection in the tree analysis was reported for each gene. All reported P-values were two-sided. All analyses were performed using the SAS statistical package version 9.2 (SAS Institute Inc., Cary, N.C., USA) and rPART function in Splus 7.0.

In this embodiment examining the correlation of CSC associated gene expression with prostate cancer recurrence after prostatectomy, 249 out of 276 men (90%) had evaluable mRNA gene expression after quality control procedures and were included in the current analysis.

Baseline Characteristics.

Table 2 shows the baseline characteristics. Cases and controls were similar with regard to median age, D'Amico risk group, and pathologic TNM stage (all comparisons P>0.05).

### Results

Individual Cancer Stem-Like Cell Gene Expression and Disease Recurrence.

Table 3 shows the results of the univariable analysis examining mRNA levels stratified by disease recurrence status. Wilcoxon analysis showed that four genes (Axin2, p<0.001; CD44a, p=0.036; OCT 4, p=0.017; TACSTD2, p=0.008) were expressed at lower levels in tumors of patients whose disease recurred.

Interdependent Cancer Stem-Like Cell Gene Expression and Disease Recurrence.

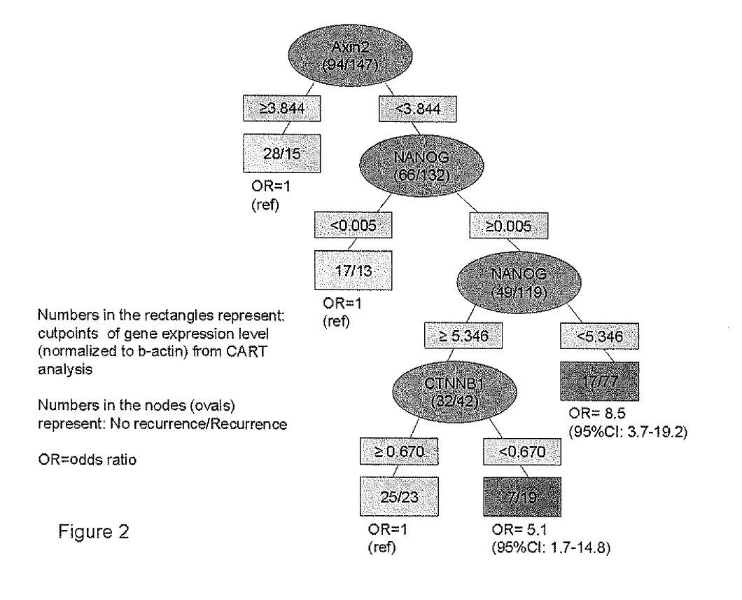

Recursive partitioning analysis was performed to identify any gene that may not be predictive alone (and thus not featured in univariable analysis) but, in association with other genes, may be associated with disease recurrence. FIG. 2 presents the CART diagram. The CSC-associated gene expression levels of Axin2, NANOG, and CTNNB1 were identified as joint determinants for disease recurrence with cut-points yielding odds ratios (OR) of 8.5 (Axin2^{low}/NANOG^{high}; n=94, 95% CI 3.7-19.2) and 5.1 (Axin2^{low}/NANOG^{very high}/CTNNB1^{low}; n=26, 95% CI 1.7-14.8).

Internal Validation.

Bootstrap internal validation was performed for the genes identified by the univariable and multivariable analysis. The consistency of these findings was supported by bootstrap analysis that selected these transcripts in more than half of the bootstrap samples for disease recurrence (Wilcoxin Analysis: Axin2, 95 out of 100; CD44a, 53 out of 100; OCT 4, 64 out of 100; TACSTD2, 70 out of 100; RP Analysis: TBD; Table 4.

### EXAMPLES

The following examples are provided in order to demonstrate and further illustrate certain embodiments and aspects of the present invention and are not to be construed as limiting the scope thereof. While such examples are typical of those that might be used, other procedures known to those skilled in the art may alternatively be utilized. Indeed, those of ordinary skill in the art can readily envision and produce further embodiments, based on the teachings herein, without undue experimentation.

### Example 1

An example of CSC-associated gene expression measurement to determine prostate cancer prognosis is described in detail above. Based on the results we observed in this particular disease setting, future patients would undergo radical prostatectomy, and their tumor tissue would be similarly assessed for the expression of Axin2, Nanog, and CTNNB1. Men with an expression profile of Axin2^{low}/NANOG^{high} or Axin2^{low}/NANOG^{very high}/CTNNB1^{low} would be considered at high risk of prostate cancer recurrence after their prostatectomy and given recommendations for more conservative management such as more frequent and intense surveillance (e.g. physical exam, CT scan, trans-rectal ultrasound, bone scan) or even adjuvant treatment with radiotherapy, hormonal therapy, or chemotherapy in an investigational setting.

### Example 2

CSC-associated gene expression measurement may be applied to additional prostate cancer disease states. For instance, in a man with serologic progression (PSA rise) after local definitive radiotherapy to the prostate, a new biopsy can be obtained from the locally-recurrent prostate tumor; this tissue would undergo mRNA extraction and qRT-PCR for Axin2/Nanog/CTNNB1. Similarly, tumor biopsy and qRT-PCR assessment of these genes could be undertaken in a man with metastatic hormone sensitive prostate cancer or in a man with metastatic castration resistant prostate cancer. In each case, the gene expression profile may be yield prognostic value for overall survival or progression-free survival.

### Example 3

CSC-associated gene expression measurement may be applied not only for prognostic purposes but also as a predictive biomarker; that is, the gene signature may be predictive of response to therapy similarly to its prediction of recurrence after prostatectomy in Example 1. In other disease states such as those described in Example 2, the Axin2/Nanog/CTNNB1 expression signature may be predictive of degree and duration of response to hormone therapy (in the serologic recurrence or metastatic hormone sensitive states) or response to chemotherapy or other targeted agents in the metastatic castration resistant state.

### Example 4

CSC-associated gene expression measurement may be applied to other cancer types, especially malignancies in which CSC subpopulations already have been described, such as gliomas, breast cancer, pancreatic cancer, liver cancer, bladder cancer, colon, and lung cancer. [R21 REFS2-7]. In these settings, biopsies from primary tumors (or metastatic sites) would be subjected to mRNA extraction and qRT-PCR quantitation of CSC-associated genes, and these measurements would yield prognostic and/or predictive information about disease outcome (e.g. recurrence, overall survival) and response to therapy.

### Example 5

If CSC-associated gene expression measurement is applied to other diseases or settings—such as other disease states in prostate cancer (Examples 2/3) or other malignancy types (Example 4)—the most highly prognostic or predictive CSC associated gene signature may prove to consist of a different combination of the CSC associated genes that we assayed. For instance, the particular biology of breast tumors may result in an Axin2^{high}/Nanog^{low} signature in contrast to the one we obtained when we assayed primary prostate tumors and correlated with prostate cancer recurrence.

### Example 6

Although CSC-associated gene expression in Example 1 and in the Detailed Description employed the mRNA extraction and qRT-PCR methods developed at Response Genetics, Inc. (RGI), the methods described in this invention do not rely upon nor are specific to those used specifically at RGI. Currently, there are multiple commercially available kits (Qiagen, Life-Tech, others) for mRNA extraction from formalin-fixed paraffin-embedded (FFPE) tumor tissue, and these kits can be used as effectively to measure and compare gene expression by qRT-PCR from a given set of tumor specimens. Moreover, the same mRNA extraction a and qRT-PCR techniques also can be applied to freshly-resected tumor tissue or to fresh-frozen tumor tissue or even to circulating tumor cells (CTCs) captured from blood samples.

### Example 7

In this example, we briefly discuss diagnostic tests, kits, devices and systems that may be implemented based on the panel of CSC-associated signature genes disclosed herein. Those skilled in the art will appreciate that the key element of any kits, devices, and systems in accordance with this invention is the CSC-associated signature genes identified in accordance with the general method discussed above. Use of the panel of signature genes will be similar to other genetic based diagnostic tests such as Oncotype DX for detection of is a diagnostic test that helps identify which women with early-stage, estrogen-receptor positive and lymph-node-negative breast cancer are more likely to benefit from adding chemotherapy to their hormonal treatment. This test also helps assess the likelihood that an individual woman's breast cancer will return. The Oncotype DX test provides important information that the patient and the doctor may use when making decisions about treatment. Post-menopausal women recently diagnosed with node-positive, hormone-receptor-positive breast cancer may also be appropriate candidates for the Oncotype DX test. (See A Multigene Assay to Predict Recurrence of Tamoxifen-Treated, Node-Negative Breast Cancer N Engl J. Med. 2004; 351(27):2817-2826, the entire content thereof is incorporated herein by reference).

Thus, it is contemplated that the test may be in the form of a RT PCR reagent kit comprising RT-PCR enzyme mix (reverse transcriptases and Taq DNA polymerase), RT-PCR buffer, dNTP mix, RNase-free water, and instruction booklet. Alternatively, the test may utilize antibody detection of proteins encoded by the genes. The test may be perform either on-site where patient tissue sample is taken or, more preferably, off-site in a diagnostic lab.

Within the diagnostic lab, there may be instruments configured to perform the test. Such instrument may be manually operated PCR machine or automated high-throughput instruments such as the SmartCycler system sold by Cepheid (Sunnyvale, Calif.). Also contemplated are reaction vials, cartridges and containers configured to house reagents and samples for automated processing.

As in the case of Oncotype DX, diagnostic and prognostic tests of the present invention may be used along or in conjunction with other clinical tests or information to guide individualized treatment.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

TABLE 1
Twelve CSC-associated genes for gene expression analysis

ALDH1A1
Axin2
Bmi1
CD133
CD44a
CTNNB1/β-catenin
ITGA2/integrin α2
NANOG
Nkx3-1
Notch1
OCT 4
TACSTD2/Trop2

TABLE 2
Baseline clinical and histological characteristics of cases (recurrences)
and control (no disease recurrence) in the tested cohort of men who
underwent prostatectomy for localized prostate cancer.

| | Recurrence-Free | Recurrence |
|---|---|---|
| | (n = 94) | (n = 147) |
| Age, yrs | | |
| Median (range) | 63 (43-78) | 64 (42-78) |
| D'Amico risk clarification | | |
| Low | 25 (27%) | 35 (24%) |
| Intermediate | 39 (41%) | 65 (44%) |
| High | 30 (32%) | 47 (32%) |
| Year of prostatectomy | | |
| 1990-1995 | 25 (27%) | 58 (39%) |
| 1996-2000 | 30 (32%) | 47 (32%) |
| 2001-2005 | 39 (41%) | 42 (29%) |

TABLE 3
Univariable analysis examining mRNA levels stratified by disease recurrence status

| | Recurrence-Free | | | Recurrence | | | |
|---|---|---|---|---|---|---|---|
| Gene | N | Median | Range | N | Median | Range | P value* |
| ALDH1A1 | 88 | 0.010 | 0.001-45.908 | 122 | 0.416 | 0.001-58.435 | 0.82 |
| Axin2 | 86 | 2.476 | 0.001-8.812 | 146 | 1.558 | 0.001-25.316 | <0.001 |
| Bmi1 | 88 | 2.541 | 0.001-13.726 | 141 | 2.802 | 0.001-17.036 | 0.34 |
| CD133 | 91 | 0.001 | 0.001-6.568 | 147 | 0.001 | 0.001-3.031 | 0.53 |
| CD44 | 79 | 1.848 | 0.001-10.102 | 136 | 1.381 | 0.001-30.536 | 0.036 |
| CTNNB1 | 91 | 0.705 | 0.001-2.957 | 138 | 0.596 | 0.001-2.611 | 0.28 |
| ITGA2 | 91 | 0.001 | 0.001-1.789 | 147 | 0.001 | 0.001-2.169 | 0.31 |
| NANOG | 75 | 5.965 | 0.001-31.189 | 111 | 3.766 | 0.001-81.742 | 0.22 |
| Nkx3 1 | 86 | 24.642 | 0.001-109.928 | 141 | 27.315 | 3.223-184.399 | 0.36 |
| Notch1 | 91 | 0.001 | 0.001-0.641 | 147 | 0.001 | 0.001-0.572 | 0.30 |
| OCT 4 | 71 | 0.001 | 0.001-8.145 | 110 | 1.005 | 0.001-14.404 | 0.017 |
| TACSTD2 | 80 | 86.601 | 19.276-251.149 | 139 | 65.594 | 8.006-293.531 | 0.008 |

TABLE 4
Results of bootstrap analysis performed for internal validation
of univariable (left) and CART analyses. For each signature,
the data set was re-sampled 100 times.

| Gene | N* | N¶ |
|---|---|---|
| ALDH1A1 | 6 | 11 |
| Axin2 | 95 | 71 |
| Bmi1 | 14 | 24 |
| CD133 | 10 | 1 |
| CD44 | 53 | 19 |
| CTNNB1 | 18 | 8 |
| ITGA2 | 19 | 0 |
| NANOG | 23 | 24 |
| Nkx3 1 | 23 | 25 |
| Notch1 | 18 | 8 |
| OCT 4 | 64 | 48 |
| TACSTD2 | 70 | 47 |

*The number of times (out of 100 bootstrap samples) that a significant association (two-sample Wilcoxon rank-sum test p value < 0.05) was observed in the univariate analysis.
¶The number of times (out of 100 bootstrap samples) that the gene was selected as being associated with outcome in the recursive partitioning analysis.

### REFERENCES

All of the references disclosed herein, including all following references are incorporated herein by reference in their entirety.
1. Siegel R, Ward E, Brawley O, Jemal A. Cancer statistics, 2011: the impact of eliminating socioeconomic and racial disparities on premature cancer deaths. CA Cancer J. Clin. 2011; 61(4):212-36.
2. Gangemi, R., Paleari, L., Orengo, A. M., Cesario, A., Chessa, L., Ferrini, S., and Russo, P. Cancer stem cells: a new paradigm for understanding tumor growth and progression and drug resistance. Curr Med Chem, 16: 1688-1703, 2009.
3. Akhtar, K., Bussen, W., and Scott, S. P. Cancer stem cells—from initiation to elimination, how far have we reached?(Review). Int J Oncol, 34: 1491-1503, 2009.
4. Gupta, P. B., Chaffer, C. L., and Weinberg, R. A. Cancer stem cells: mirage or reality? Nat Med, 15: 1010-1012, 2009.
5. Park, C. Y., Tseng, D., and Weissman, I. L. Cancer stem cell-directed therapies: recent data from the laboratory and clinic. Mol Ther, 17: 219-230, 2009.
6. O'Brien, C. A., Kreso, A., and Dick, J. E. Cancer stem cells in solid tumors: an overview. Semin Radiat Oncol, 19: 71-77, 2009.
7. Visvader, J. E. and Lindeman, G. J. Cancer stem cells in solid tumours: accumulating evidence and unresolved questions. Nat Rev Cancer, 8: 755-768, 2008.
8. Desai B, Rogers M J, Chellaiah M A. Mechanisms of osteopontin and CD44 as metastatic principles in prostate cancer cells. Mol Cancer. 2007 Mar. 7; 6:18.
9. Mani S A, Guo W, Liao M J, et al. The epithelial-mesenchymal transition generates cells with properties of stem cells. Cell. 2008 May 16; 133(4):704-15.
10. Ben-Porath I, Thomson M W, et al. An embryonic stem cell-like gene expression signature in poorly differentiated aggressive human tumors. Nat. Gen. 2008 May. 40(5): 499-507.
11. Chiou S H, Yu C C, et al. Positive Correlations of Oct-4 and Nanog in Oral Cancer Stem-Like Cells and High-Grade Oral Squamous Cell Carcinoma. Clin Cancer Res 4085 2008; 14(13) Jul. 1, 2008.
12. Maeda S, Shinchi H, et al. CD133 expression is correlated with lymph node metastasis and vascular endothelial growth factor-C expression in pancreatic cancer. British Journal of Cancer (2008) 98, 1389-1397.
13. Mylona E, Giannopoulou I, et al. The clinicopathologic and prognostic significance of CD44+/CD24−/low and CD44−/CD24+ tumor cells in invasive breast carcinomas. Human Pathology (2008) 39, 1096-1102.
14. Rebetz J, Tian D, et al. Glial Progenitor-Like Phenotype in Low-Grade Glioma and Enhanced CD133-Expression and Neuronal Lineage Differentiation Potential in High-Grade Glioma. PLoS ONE 2008 April, 3(4) 1-12.
15. Song W, Li H, et al. Expression and clinical significance of the stem cell marker CD133 in hepatocellular carcinoma. Int J Clin Pract, August 2008, 62, 8, 1212-1218,
16. Zeppernick F, Ahmadi R, et al. StemCell Marker CD133 Affects Clinical Outcome in Glioma Patients. Clin Cancer Res 2008; 123 14(1) Jan. 1, 2008.
17. Gotte M, Wolf M, et al. Increased expression of the adult stem cell marker Musashi-1 in endometriosis and endometrial carcinoma. J Pathol 2008; 215: 317-329.
18. Woo H G, Wang X W, et al. Association of TP53 Mutations With Stem Cell-Like Gene Expression and Survival of Patients With Hepatocellular Carcinoma. GASTROENTEROLOGY 2011; 140:1063-1070.
19. National Comprehensive Cancer Network: NCCN Clinical Practice Guidelines in Oncology: Prostate Cancer Version 1.2012. Available at www.nccn.org. Accessed Mar. 1, 2012.
20. Stephenson A J, Scardino P T, Eastham J A, et al: Postoperative nomogram predicting the 10-year probability of prostate cancer recurrence after radical prostatectomy. J Clin Oncol 23:7005-7012, 2005.
21. Stephenson A J, Kattan M W, Eastham J A, et al: Prostate cancer-specific mortality after radical prostatectomy for patients treated in the prostate-specific antigen era. J Clin Oncol 27:4300-4305, 2009.
22. Markert E K, Mizuno H, Vazquez, et al: Molecular classification of prostate cancer using curated expression signatures. Proc Natl Acad Sci USA 108:21276-21281, 2011.
23. Wang S Y, et al: Pten deletion leads to the expansion of a prostatic stem/progenitor cell subpopulation and tumor initiation. Proc Natl Acad Sci USA 103:1480-1485, 2006.
24. Tomlins S A, Rhodes D R, Perner S, et al: Recurrent fusion of TMPRSS2 and ETS transcription factor genes in prostate cancer. Science 310:644-648, 2005.
25. Demichelis F, Fall K, Perner S, et al: TMPRSS2:ERG gene fusion associated with lethal prostate cancer in a watchful waiting cohort. Oncogene 26:4596-4599, 2007.
26. Macaluso M, Giordano A: TMPRSS2:ERG Gene Fusion: a new genetic marker for prostate cancer progression. Cancer Biol Ther 6:46-47, 2007.
27. Schlomm T, et al: Clinical significance of p53 alterations in surgically treated prostate cancers. Mod Pathol 21:1371-1378, 2008.
28. Jenkins R B, Qian J, Lieber M M, et al: Detection of c-myc oncogene amplication and chromosomal anomalies in metastatic prostatic carcinoma by fluorescence in situ hybridization. Cancer Res 57:524-531, 1997.
29. Rubin M A, Bismar T A, Andren O, et al: Decreased alpha-methylacyl CoA racemase expression in localized prostate cancer is associated with an increased rate of biochemical recurrence and cancer-specific death. Cancer Epidemiol Biomarkers Prev 14:1424-1432, 2005.
30. Bismar T A, Demichelis F, Riva A, et al: Defining aggressive prostate cancer using a 12-gene model. Neoplasia 8:59-68, 2006.
31. Glinsky G V, Glinskii A B, Stephenson A J, et al: Gene expression profiling predicts clinical outcome of prostate cancer. J Clin Invest 113:913-923, 2004.
32. Singh D, Febbo P G, Ross K, et al: Gene expression correlates of clinical prostate cancer behavior. Cancer Cell 1:203-209, 2002.
33. Cheville J C, Karnes R J, Therneau T M, et al: Gene panel model predictive of outcome in men at high-risk of systemic progression and death from prostate cancer after radical retropubic prostatectomy. J Clin Oncol 26:3930-3935, 2008.
34, Penney K L, Sinnott J A, Fall K, et al: mRNA expression signature of Gleason grade predicts lethal prostate cancer. J Clin Oncol 29:2391-2396, 2011.
35. Shen M M, Abate-Shen C: Molecular genetics of prostate cancer: new prospects for old challenges. Genes Dev 24:1967-2000, 2010.
36. Dorin R P, Daneshmand S, Lassoff M A, et al: Long-term outcomes of open radical retropubic prostatectomy for clinically localized prostate cancer in the prostate-specific antigen era. Urology 2012 In press.
37. Lord R V, Brabender J, Gandara D, et al: Low ERCC1 expression correlates with prolonged survival after cisplatin plus gemcitabine chemotherapy in non-small cell lung cancer. Clin Cancer Res 8:2286-2291, 2002.

What is claimed is:

1. A method of formulating a cancer diagnostic or prognostic test, comprising:
selecting a set of candidate cancer stem cell (CSC)-associated genes;
determining expression levels of said candidate CSC-associated genes in tissues samples obtained from a population consisting of a control group and a comparison group;
analyzing the expression levels of the candidate genes to identify one or more diagnostic gene(s) that show differential expression patterns in the control and test groups; and
formulating the diagnostic or prognostic test using the expression levels of the diagnostic gene(s) as input.

2. The method of claim 1, wherein said analyzing step further comprising a univariable analysis, a multivariable analysis, or both.

3. The method of claim 2, wherein said multivariable analysis is classification and regression tree based on recursive partitioning.

4. The method of claim 1, wherein said formulating step further comprising a step of generating a receiver operating curve for the test.

5. The method of claim 1, wherein said cancer is selected from the group consisting of prostate, breast, lung, colon, bladder, liver, pancreatic and brain cancer and the tissue sample preferably comprises at least a portion of tumor tissue thereof.

6. The method of claim 1, wherein said set of candidate CSC-associated genes consist of ALDH1A1, Axin2, Bmi1, CD133, CD44a, CTNNB1/β-catenin, ITGA2/integrin α2, NANOG, Nkx3-1, Notch1, OCT 4, TACSTD2/Trop2.

7. The method of claim 1, wherein said diagnostic gene(s) are selected from the group consisting of Axin2, NANOG, and CTNNB1.

8. The method of claim 1, wherein said diagnostic gene(s) is Axin2.

9. The method of claim 1, wherein said diagnostic gene(s) are selected from the group consisting of Axin2, CD44, Oct4, and TACSTD2.

10. The method of claim 1, wherein said cancer is prostate cancer, said control group is subjects who have non-recurring prostate cancer post prostatectomy and the comparison group is subjects who have recurring prostate cancer post prostatectomy.

11. The method of claim 1, wherein said expression levels of the gene(s) are determined at the mRNA level, the protein level, or combination thereof.

12. A method of formulating a diagnostic/prognostic test for cancer, wherein said diagnostic/prognostic test having the values of a predetermined set of CSC-associate gene(s) as its parameters, said method comprising:
making a diagnosis/prognosis determination on a test data set of CSC-associate gene(s) expression levels, wherein said data set having a plurality of control and comparison data points, said determination is based on an initial set of cutoff values for the expression levels;
generating a receiver operating characteristic (ROC) curve by varying the cutoff values;
selecting an optimal point along the ROC curve; and
choosing the cutoff values corresponding to the optimal point as the final values for the diagnostic/prognostic test.

13. A diagnostic/prognostic assay kit for determining a likelihood of prostate cancer recurrence after prostatectomy, comprising:
reagents for determining expression levels of a predetermined set of CSC-associated gene(s); and
an instruction insert with printed instructions for directing a user to perform the test and interpret the result.

14. The kit of claim 13, wherein said CSC-associated gene(s) are selected from the group consisting of Axin2, CD44, Oct4, TACSTD2, NANOG, and CTNNB1.

15. The kit of claim 13, wherein said CSC-associated gene(s) are selected from the group consisting of Axin2, CD44, Oct4, and TACSTD2.

16. The kit of claim 13, wherein said CSC-associated gene(s) are selected from the group consisting of Axin2, NANOG, and CTNNB1.

17. The kit of claim 13, wherein said CSC-associated gene(s) include at least Axin2.

18. A method of cancer prognosis or prediction in a test subject, the method comprising:
providing a tissue sample from the test subject, the tissue sample comprising a population of cancer cells;
measuring an expression level of at least one cancer stem cell associated (CSC-associated) gene in the tissue sample;
applying the test formed by method 1 or 12 to the expression level; and
based on the result of the test, identifying the test subject as either likely or unlikely to experience a clinical response.

19. A method for predicting the likelihood of recurrence of prostate cancer following prostatectomy in a test subject, the method comprising:
providing a tissue sample from the test subject, the tissue sample comprising a population of prostate cancer cells;
measuring an expression level of at least one cancer stem cell associated (CSC-associated) gene in the tissue sample;
comparing the expression level by comparing the expression level of the at least one CSC-associated gene measured in the test sample to a reference expression level having derived from a cohort of patients who have not experienced recurrence of prostate cancer following prostatectomy; and
identifying the test subject as either likely to experience a recurrence of prostate cancer based on the comparison.

20. The method according to claim 19, wherein the tissue sample selected from tumor tissue, snap frozen tissues, isolated circulating tumor cells, desiccated samples, freshly resected samples and freshly frozen samples.

21. The method of according to claim 19, wherein the tissue sample is a fixed formalin tissue sample.

22. The method according to claim 19, wherein the at least one CSC-associated gene is selected from the group consisting of ALDH1A1, Axin2, Bmi1, CD133, CD44s, CTNNB1/β-catenin, ITGA2/integrin α2, NANOG, Nkx3-1, Notch1, OCT4, TACSTD2/Trop2.

23. The method according to claim 22, wherein the at least one CSC-associated gene is selected from the group consisting of Axin2, NANOG, and CTNNB1.

24. The method according to claim 23, wherein the at least one CSC-associated gene is Axin2.

25. The method of claims 19, further comprising the steps of:
microdissecting areas of tissue sample selected from formalin fixed paraffin embedded (FFPE) tumor tissue specimens, snap frozen samples, isolated circulating tumor cells, desiccated samples, freshly resected samples and freshly frozen samples;
extracting biomarkers from the microdissected tumor tissue, wherein said biomarkers are selected from mRNA transcripts of the signature genes, proteins encoded by the mRNA, and a combination thereof;
measuring levels of the biomarker with a suitable analytical technique; and
corresponding said biomarker levels to the expression levels of the CSC-associated signature gene(s), normalizing to housekeeping genes where necessary.

26. The method of claims 2, further comprising the steps of:
microdissecting areas of tissue sample selected from formalin fixed paraffin embedded (FFPE) tumor tissue specimens, snap frozen samples, isolated circulating tumor cells, desiccated samples, freshly resected samples and freshly frozen samples;
extracting biomarkers from the microdissected tumor tissue, wherein said biomarkers are selected from mRNA transcripts of the signature genes, proteins encoded by the mRNA, and a combination thereof;
measuring levels of the biomarker with a suitable analytical technique; and
corresponding said biomarker levels to the expression levels of the CSC-associated signature gene(s), normalizing to housekeeping genes where necessary.
